(19) United States
(12) Patent Application Publication
(10) Pub. No.: US 2021/0128299 A1
(43) Pub. Date: May 6, 2021

(54) SYSTEMS AND METHODS FOR DEPLOYING TRANSCATHETER HEART VALVES

(71) Applicant: Bahram Khadivi, Rancho Santa Fe, CA (US)

(72) Inventor: Bahram Khadivi, Rancho Santa Fe, CA (US)

(21) Appl. No.: 17/083,169

(22) Filed: Oct. 28, 2020

Related U.S. Application Data
(63) Continuation of application No. 62/927,120, filed on Oct. 28, 2019.

(51) Int. Cl. A61F 2/24; A61B 90/00; A61M 25/01

(52) U.S. Cl. CPC A61F 2/2427; A61B 2090/376; A61M 25/0108; A61B 90/37

(57) ABSTRACT

Systems and methods for positioning a transcatheter aortic valve are disclosed. A system can include a first catheter, a second catheter, and a flexible wire all independently movable with respect to each other, and configured to be implanted in respective aortic valve cusps in order to accurately determine a real-time coplanar angle of the nadirs of each of the first aortic valve cusp, the second aortic valve cusp, and the third aortic valve cusp, thereby determining a desired valve implantation depth.

### PRIORITY CLAIM

This application claims the benefit under 35 U.S.C. § 119(e) as a nonprovisional application of U.S. Pat. App. No. 62/927,120 filed on Oct. 28, 2019, which is hereby incorporated by reference in its entirety.

### BACKGROUND

The aortic valve controls blood flow from the left ventricle to the aorta where oxygenated blood is diverted throughout the systemic vasculature. The aortic valve has three relatively symmetric leaflets in the majority of people. Degenerative valvular heart disease leads to slow destruction and/or calcification of these leaflets, resulting in aortic valve stenosis or regurgitation. Transcatheter heart valve implantation (sometimes referred to as either TAVR—transcatheter aortic valve replacement, or TAVI—transcatheter aortic valve implantation) has become mainstream therapy for intermediate and high surgical risk patients, now also expanding to low surgical risk patients. TAVR is performed by implanting a transcatheter heart valve (THV) across the previously degenerated native aortic valve. It can be very important to ensure appropriate implant depth relative to the native aortic valve annulus. This can advantageously minimize the risk of device embolism, permanent pacemaker placement due to conduction system disturbance, and paravalvular regurgitation involving the newly implanted valve.

The aortic annulus has many definitions, but in TAVR therapy, it can be defined as the nadir of the three aortic valve cusps in a coplanar angle where the nadir of the three cusps are equidistant and parallel to each other. This coplanar angle can be approximated in most cases by a computerized tomography (CT) scan as part of pre-procedural evaluation for TAVR. However, there are limitations to this approach, including but not limited to: 1) patient position on the CT scan table may not be the same as during TAVR leading to an inaccurate coplanar angle; 2) CT image quality may not be adequate for obtaining an accurate coplanar angle; 3) not all patients can tolerate the amount of iodinated contrast necessary for accurate CT scan imaging—this is typically due to chronic kidney disease. As a result, implanting TAVR physicians often have to make fine or sometimes major adjustments with repeated aortic root angiograms to obtain the appropriate angle immediately prior to THV implant; this can lead to excessive radiation to the patient and physician as well as excessive iodinated contrast use and consequent kidney failure risk. In cases of severe aortic regurgitation, obtaining a coplanar angle by traditional angiography method described may not be possible due to brisk wash out of iodinated contrast from the aortic root due to a significant regurgitant jet. Improved systems and methods for accurately deploying transcatheter heart valves with respect to the native valve annulus are needed.

### SUMMARY

In some embodiments, systems and methods as disclosed herein can include a catheter with a distal segment or segments configured to contact, be proximate to, and/or locate the nadir or near-nadir of two aortic valve cusps. Catheters can also be configured for use with angiography as clinically necessary. The catheter can contact the nadir of the cusp or at least be in the vicinity in patients with smaller aortic sinus/cusps.

In some embodiments a system or catheter can include any number of features as disclosed herein.

In some embodiments, a curved section of a catheter can be configured to contact the lesser curve or the greater curve of the ascending aorta. The portion of the catheter that sits across the aortic arch can be configured to contact the lesser curve of the aorta arch, the greater curve of the aorta arch, or be “free-floating” in the aortic arch depending on manipulation necessary to obtain proper distal catheter position in the aortic cusp of interest.

In some embodiments, disclosed herein is a method for positioning a transcatheter aortic valve, comprising any number of: positioning a first catheter in a first aortic valve cusp; positioning a second catheter in a second aortic valve cusp, the second catheter not connected to the first catheter; positioning a flexible wire in a third aortic valve cusp, visualizing nadirs of each of the first aortic valve cusp, the second aortic valve cusp, and the third aortic valve cusp in real-time utilizing an imaging device by locating a portion of the first catheter, second catheter, and flexible wire; and manipulating the imaging device to determine a real-time coplanar angle of the nadirs of each of the first aortic valve cusp, the second aortic valve cusp, and the third aortic valve cusp, thereby determining a desired valve implantation depth.

In some embodiments, after positioning the first catheter, the second catheter, and the flexible wire each of the first catheter, the second catheter, and the flexible wire are independently movable with respect to each other.

In some embodiments, the first catheter is a pigtail catheter.

In some embodiments, the first aortic valve cusp is a right coronary cusp or a non-coronary cusp.

In some embodiments, the second aortic valve cusp is a right coronary cusp or a non-coronary cusp,

In some embodiments, the third aortic valve cusp is a left coronary cusp.

In some embodiments, at least one or both of the first catheter and the second catheter comprises a radiopaque marker.

In some embodiments, the method can also include delivering contrast media through a lumen of the second catheter.

In some embodiments, the contrast media exits the second catheter via a plurality of exit apertures along the sidewall of the second catheter.

In some embodiments, the imaging device comprises a C-arm X-ray imaging device.

In some embodiments, manipulating the imaging device comprises moving the C-arm,

In some embodiments, the imaging device is not a CT imaging device.

In some embodiments, the second catheter comprises: a proximal handle; a proximal segment; and/or a distal segment and tip.

In some embodiments, the distal segment comprises a first portion, a first semicircular arc, a second portion, a third portion, and/or a second semicircular arc,

In some embodiments, the first portion comprises a negative arc.

In some embodiments, the first portion comprises a positive arc,

In some embodiments, the second portion or a third portion comprises a negative arc.

In some embodiments, the second portion or a third portion comprises a positive arc.

In some embodiments, the first semicircular arc comprise a first central axis of which the first semicircular arc can rotate around, wherein an angle formed by the central axes and a horizontal axis is between about 20 degrees and about 150 degrees, or between about 60 degrees and about 120 degrees.

In some embodiments, the second semicircular arc comprise a second central axis of which the second semicircular arc can rotate around, wherein an angle formed by the central axes and a horizontal axis is between about −10 degrees and about −120 degrees, or between about −30 degrees and about −80 degrees.

In some embodiments, the proximal segment is a substantially straight segment.

In some embodiments, the proximal segment comprises a curved segment having an arc measure of between about 100 degrees and about 170 degrees, or between about 130 degrees and about 170 degrees.

In some embodiments, a catheter system for positioning a transcatheter aortic valve can comprise any number of: a first catheter configured to be positioned in a first aortic valve cusp; a second catheter configured to be positioned in a second aortic valve cusp, the second catheter not connected to the first catheter; and a flexible wire configured to be positioned in a third aortic valve cusp. The first catheter, second catheter, and the flexible wire can be utilized to visualize nadirs of each of the first aortic valve cusp, the second aortic valve cusp, and the third aortic valve cusp in real-time utilizing an imaging device by locating a portion of the first catheter, second catheter, and flexible wire with respect to their proximity to or contact with each of the first aortic valve cusp, the second aortic valve cusp, and the third aortic valve cusp. After positioning the first catheter, the second catheter, and the flexible wire each of the first catheter, the second catheter, and the flexible wire can be independently movable with respect to each other.

In some embodiments, the first catheter is a pigtail catheter,

In some embodiments, at least one or both of the first catheter and the second catheter comprises a radiopaque marker.

In some embodiments, the second catheter comprises: a proximal handle; a proximal segment; and/or a distal segment and tip.

In some embodiments, the distal segment comprises a first portion, a first semicircular arc, a third portion, and a second semicircular arc.

In some embodiments, the first portion comprises a negative arc.

In some embodiments, the first portion comprises a positive arc.

In some embodiments, the second portion comprises a negative arc.

In some embodiments, the second portion comprises a positive arc.

In some embodiments, the first semicircular arc comprises a first central axis of which the first semicircular arc can rotate around, wherein an angle formed by the central axes and a horizontal axis is between about 20 degrees and about 150 degrees, or between about 60 degrees and about 120 degrees.

In some embodiments, the second semicircular arc comprise a second central axis of which the second semicircular arc can rotate around, wherein an angle formed by the central axes and a horizontal axis is between about −10 degrees and about −120 degrees, or between about −30 degrees and about −80 degrees.

In some embodiments, the proximal segment is a substantially straight segment.

In some embodiments, the proximal segment comprises a curved segment having an arc measure of between about 100 degrees and about 170 degrees, or between about 130 degrees and about 170 degrees.

In some embodiments, a system for positioning an aortic valve can comprise, consist essentially of, consist of, and/or not comprise any number of features of the disclosure.

In some embodiments, a method for positioning an aortic valve can comprise, consist essentially of, consist of, and/or not comprise any number of features of the disclosure.

### BRIEF DESCRIPTION OF THE DRAWINGS

FIG. 1 schematically illustrates an embodiment of an implant positioning system.

FIG. 2 schematically illustrates the implant positioning system of FIG. 1, with arrows illustrating left anterior oblique (LAO) and cranial (CRA) projection until all three cusp markers are coplanar.

FIG. 3 schematically illustrates the implant positioning system of FIGS. 1-2 after appropriate manipulation of an imaging apparatus,

FIG. 4 schematically illustrates the distal segment/tip portion of an elongate member, such as a catheter, including non-limiting dimensions and other specifications, according to some embodiments of the invention.

FIG. 5 schematically illustrates additional features of the distal segment/tip portion of the catheter of FIG. 4, according to some embodiments,

FIGS. 6A-6C schematically illustrate various catheter geometries, according to some embodiments of the invention.

FIG. 7 schematically illustrates the distal end of an elongate member, such as a catheter including one, two, or more radiopaque marker elements.

FIG. 8 schematically illustrates the distal end of an elongate member, such as a catheter including a plurality of axially regularly or irregularly spaced-apart side apertures configured to allow for the simultaneous injection of contrast media.

FIG. 9 schematically illustrates that the distal tip of the catheter may take a 3-dimensional geometry in some embodiments, in that the distal tip can have an angle clockwise or counterclockwise to the long axis of the catheter.

FIGS. 10A-10B schematically illustrate embodiments of application of the “Follow the Right Cusp” rule in normal and horizontal aortic root anatomy, respectively.

### DETAILED DESCRIPTION

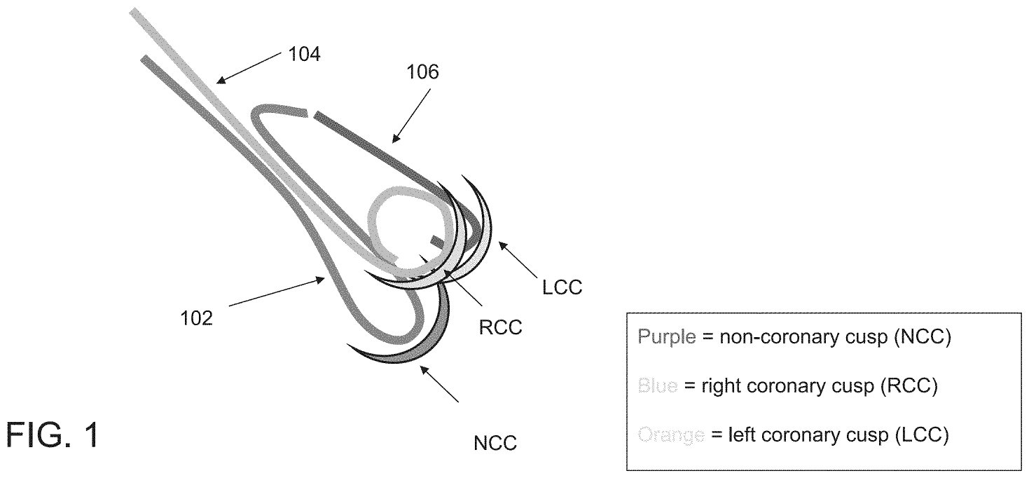

In some embodiments, catheter systems and methods are described which assist in appropriate positioning of the X-ray imaging C-arm in obtaining a coplanar angle during a valve replacement procedure, such as a TAVR procedure for example. A catheter can include, for example, a hollow tube made of plastic or another biocompatible material through which a wire can be passed therethrough. The wire can, in some embodiments, include a soft, non-rigid atraumatic distal end and tip in addition to a stiffer body, allowing catheter straightening when the rigid part of the wire is extended to provide atraumatic delivery of the catheter retrograde through the vasculature to the aortic valve/root complex. Once this wire is retracted to the flexible, non-rigid segment, the catheter transforms to its previous configuration and can be positioned in a stable manner in one of the aortic valve cusps—this is generally the non-coronary cusp or the right coronary cusp, although it could also be the left coronary cusp. The flexible wire can then be directed deep into another aortic valve cusp (this is generally the left coronary cusp, although could be one of the other aortic valve cusps) by advancing the wire and having it curl in the respective cusp. The catheter can be rotated in an appropriate direction, e.g., clockwise or counterclockwise, to aid in advancing the wire into the appropriate position within the other cusp. A TAVR procedure very commonly requires a standard pigtail catheter be present within one of the aortic valve cusps (this is generally the right coronary cusp or non-coronary cusp) to allow for angiography prior to valve implantation. Having the base of a catheter in one aortic valve cusp (for example, the non-coronary cusp) and the flexible, non-rigid wire in another aortic valve cusp (for example, the left coronary cusp) along with a previously mentioned standard pigtail catheter in the remaining aortic valve cusp (for example, the right coronary cusp) allows real time visualization of the nadirs of each of the aortic valve cusps under fluoroscopy. FIG. 1 schematically illustrates an embodiment of an implant positioning system including a first elongate member 104 positioned in the right coronary cusp RCC, a second elongate member 102 in the noncoronary cusp NCC, and a third elongate member 106 positioned in the left coronary cusp LCC.

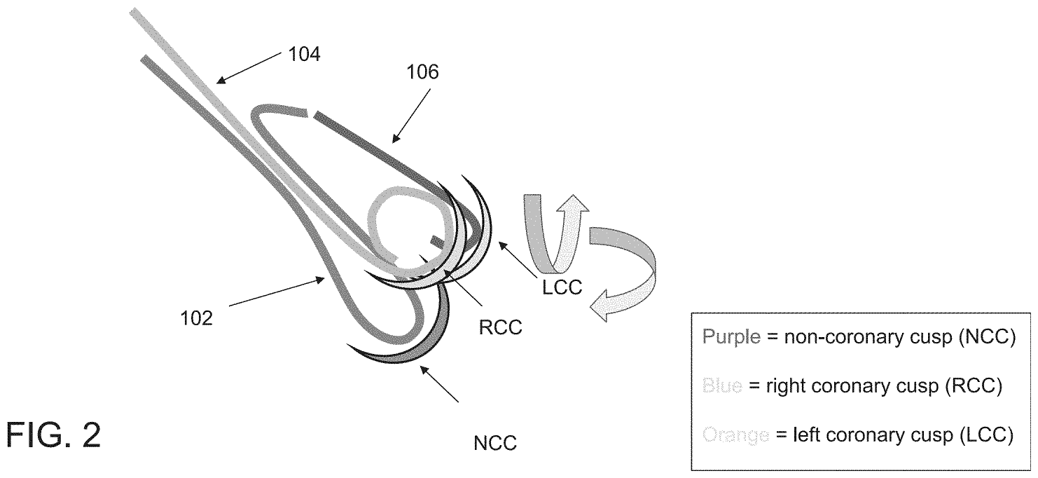

In some embodiments, each of the three elongate members, for example, a first catheter, flexible wire, and second catheter, e.g., pigtail catheter are independently movable with respect to each other, and are not directly attached to each other to allow for advantageous ease of adjustment when aligning the elongate members to obtain an accurate co-planar angle. However, in other embodiments, each of the three elongate members are directly attached to each other, such as at a common proximal hub. In some embodiments, each of the catheter, flexible wire, and pigtail catheter have different distal end geometries. In some embodiments, the pigtail catheter is placed prior to the second catheter and the flexible wire. The X-ray imaging C-arm can now be moved according to the “Follow the Right Rule” (Kasel A M et al, JACC: Cardiovasc Img 2013) to obtain a real time intra-procedure coplanar angle prior to valve implantation. FIG. 2 schematically illustrates the implant positioning system of FIG. 1, with arrows illustrating left anterior oblique (LAO) and cranial (CRA) projection until all three cusp markers are coplanar. As described above, this allows optimal valve implantation depth and minimizes risk of device embolization, conduction system disturbance and resultant permanent pacemaker implantation requirement; valve hemodynamic function is also improved with less potential for significant paravalvular regurgitation and more optimal antegrade valve flow dynamics in cases of a high implant with respect to native aortic annulus,

Relative orientation of the RCC can be utilized to adjust the flat detector angle to obtain the final coplanar angle (hence “follow the right/RCC rule”). Some methods can start with the standard pigtail catheter in the RCC (this can also allow better contrast filling of the NCC and LCC). The pigtail is taken to the NCC in some embodiments for COREVALVE (Medtronic, Inc.) and LOTUS (Boston Scientific, Inc.) valve deployment, for example, but can stay in the RCC for SAPIEN (Edwards Lifesciences, Inc.) valve deployment.

For an aortic root that is non-horizontal (“normal” aortic root) in angulation, this rule can work as follows, as a non-limiting example:

if the RCC is too cranial, then the flat detector is taken more cranial,

if the RCC is too caudal, then the flat detector is taken more caudal;

if the RCC is too rightward, then the flat detector is taken more RAO; and/or

if the RCC is too leftward, then the flat detector is taken more LAO.

For an aortic root that is horizontal in angulation, this rule can work as follows, as a non-limiting example:

if the RCC is too cranial, then the flat detector is taken more RAO;

if the RCC is too caudal, then the flat detector is taken more LAO;

if the RCC is too rightward, then the flat detector is taken more caudal; and/or

if the RCC is too leftward, then the flat detector is taken more cranial.

In some cases, the flat detector can be adjusted using a combination of the above-mentioned dual planes (RAO/LAO and CRA/CAU) to get to the final coplanar angle. Moreover, the definition of horizontal vs non-horizontal aortic root may not be entirely clear which translates clinically into the fact that above adjustments do not usually have isolated/single plane effects on obtaining the final coplanar angle. Most aortas are neither “normal” nor “horizontal,” rather they fall somewhere between these two extremes. For example, if the RCC is too leftward, then LAO corrects for this but can also take the RCC more caudal in which another adjustment may be necessary. Embodiments as disclosed herein can advantageously allow real time visualization of these changes for more efficient and precise coplanar angle attainment, and movement in any combination of the RAO, LAO, caudal, and/or cranial directions, including multiple movements in a single direction or combination of directions. In some embodiments, the Right Rule is not necessarily followed, and other adjustments may be made depending on the desired clinical result.

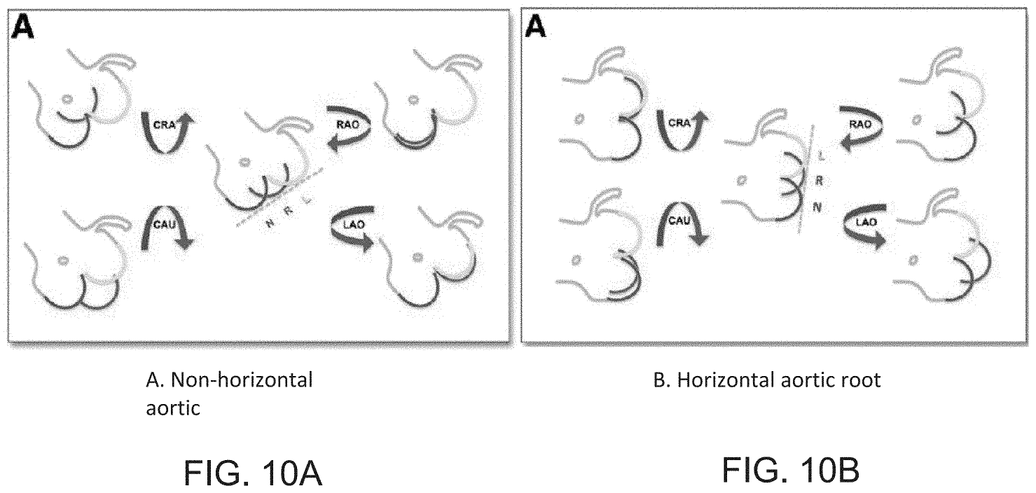

FIGS. 10A-10B schematically illustrate embodiments of application of the “Follow the Right Cusp” rule in normal (FIG. 10A) and horizontal aortic root anatomy (FIG. 10B), respectively.

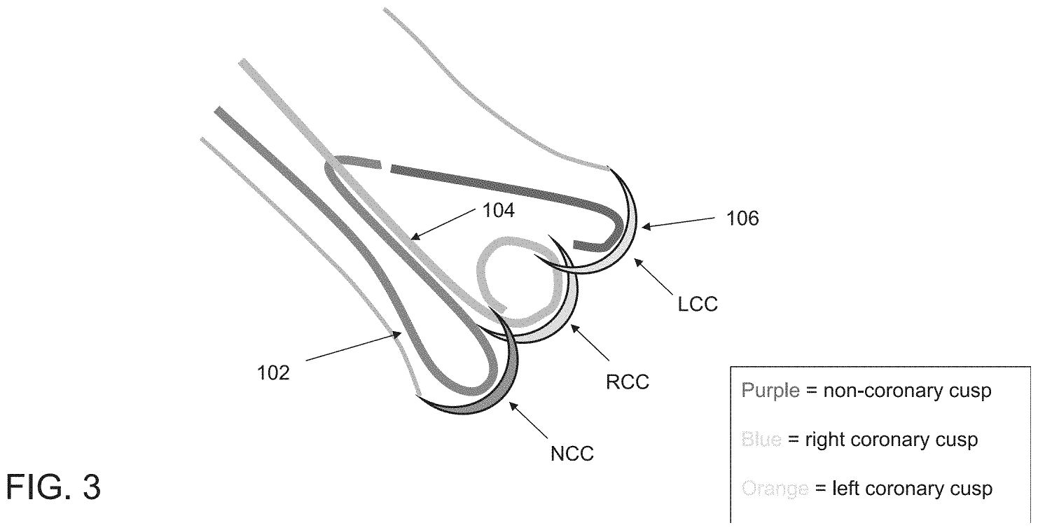

FIG. 3 schematically illustrates the implant positioning system of FIGS. 1-2 after appropriate manipulation of an imaging apparatus, such as an X-ray imaging C-arm and a coplanar angle has been obtained.

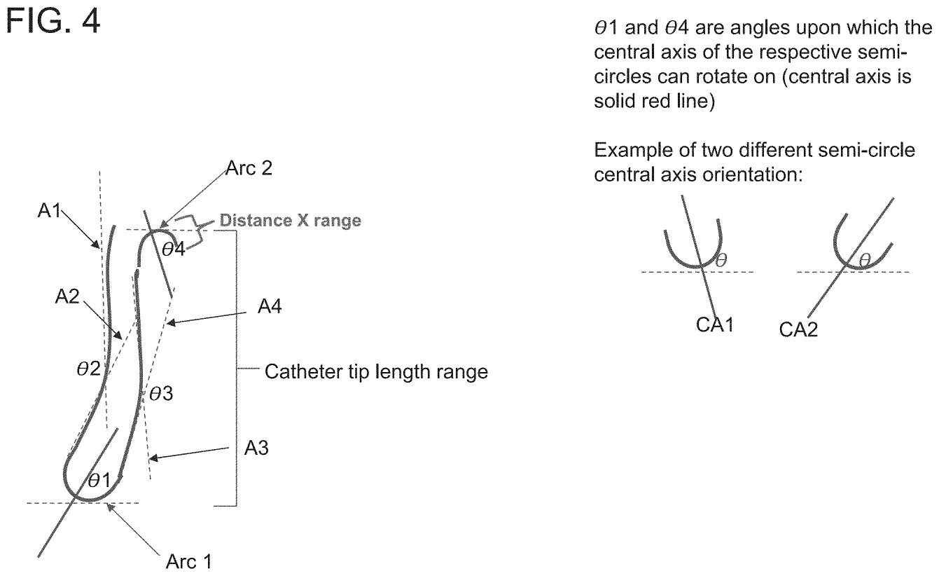

FIG. 4 schematically illustrates the distal segment/tip portion of an elongate member, such as a catheter, including non-limiting dimensions and other specifications, according to some embodiments of the invention. The particular geometries and other features of various portions of the elongate member are described herein in their unstressed, preformed configuration. Specific catheter geometries as described herein can be configured for, and especially advantageous to be placed within one or more aortic valve cusps such as a bottom portion of a distal segment of the catheter can contact or be closely proximate the nadir of the one or more aortic valve cusps sufficient to accurately measure a coplanar angle of a valve annulus such as described elsewhere herein.

The catheter can be made of any desired material, including but not limited to silicone, polyurethane (PU), polyethylene (PE), polyvinylchloride (PVC), ePTFE, PTFE, nylon, and combinations thereof. The catheter can have a biocompatible hydrophillic coating on its entire length or segments thereof. The catheter can include one, two, or more lumens, such as one or more fluid lumens, and/or a lumen configured to house a guidewire. The catheters can include proximal handles.

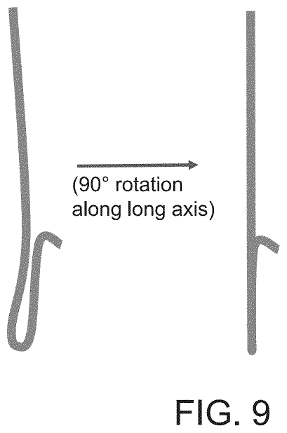

In some embodiments, the distal segment/tip portion of the elongate member can have a length of between about 20 mm and about 40 mm, such as about 20 mm, 30 mm, 40 mm, or ranges including any two of the foregoing values. The distal tip of the catheter may take a 3-dimensional geometry in some embodiments (as illustrated in FIG. 9) iii that the distal tip can have an angle clockwise or counterclockwise to the long axis of the catheter between, for example, about 0 degrees and about 45 degrees, between about 20 degrees and about 40 degrees, between about 10 degrees and about 30 degrees, or about 0, 5, 10, 15, 20, 25, 30, 35, 40, 45 degrees, or ranges including any two of the foregoing values. The arrow indicates the catheter when viewed from its long axis, demonstrating 3-dimensional geometry of catheter tip which can be angled clockwise or counterclockwise relative to its long axis for easier flexible wire advancement into another aortic valve cusp.

In some embodiments, the catheter includes a number of angled regions, from proximal to distal, θ2, θ1, θ3, and θ4. Some embodiments do not necessarily include all four angled regions, and could include only one, two, or three angled regions. Some embodiments include all four angled regions, and additional angled regions proximal or distal to the four illustrated angled regions, or in between any of the angled regions. In some embodiments, one, two, three, or four of the angled regions have different angles. As illustrated in the right half of FIG. 4, θ1 and θ4 are angles upon which the central axis of the respective arc (e.g., Arc 1 and Arc 2), such as, for example, a semi-circular or substantially semi-circular shaped arc can rotate on relative to horizontal as shown (central axes CA1 and CA2, respectively, are the solid lines). Two different non-limiting examples of different semi-circle central axis orientation are shown. In some embodiments, θ1 can be an angle of between about 20 degrees and about 150 degrees, between about 50 degrees and about 100 degrees, between about 60 degrees and about 120 degrees, between about 70 degrees and about 90 degrees, or about 20, 30, 40, 50, 60, 70, 80, 90, 100, 110, 120, 130, 140, 150 degrees, or ranges including any two of the foregoing values.

In some embodiments, Arc 1 and/or Arc 2 have a sufficient arc measure such that the segment of catheter at least somewhat “doubles back,” in other words curves around in a second direction that is at least tangentially opposite of the first direction (e.g., an arc having an arc measure of at least about 90 degrees, such as between about 90 degrees and about 180 degrees).

In some embodiments, Arc 1 can have a radius of between about 2 mm and about 9 mm, between about 4 mm and about 7 mm, between about 2 mm and about 5 mm, between about 3 mm and about 4 mm, or about 2, 3, 4, 5, 6, 7, 8, 9 mm, or ranges including any two of the foregoing values. In some embodiments, the catheter is sized and configured such that the inflection point of Arc 1 is configured to contact or substantially contact the nadir of a valve cusp, or at least be in the vicinity of the nadir of the cusp, such as less than, for example, about 5 mm, 4 mm, 3 mm, 2 mm, 1 mm, or less away from the nadir or the cusp.

In some embodiments, Arc 2 can have a radius of between about 1 mm and about 9 mm, between about 2 mm and about 5 mm, between about 4 mm and about 7 mm, or about 1, 2, 2.5, 3, 3.5, 4, 4.5, 5, 6, 7, 8, 9 mm, or ranges including any two of the foregoing values.

In some embodiments, Arc 1 can have an arc length of between about 2 mm and about 35 mm, between about 10 mm and about 26 mm, such as about, at least about, or no more than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 mm, or more or less, or ranges including any two of the foregoing values.

In some embodiments, Arc 2 can have an arc length of between about 2 mm and about 35 mm, between about 5 mm and about 20 mm, such as about, at least about, or no more than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 mm, or more or less, or ranges including any two of the foregoing values.

In some embodiments, Arc 1 can have an arc measure of between about 50 degrees and about 270 degrees, between about 80 degrees and about 220 degrees, or about 50, 60, 70, 80, 90, 100, 110, 120, 130, 140, 150, 160, 170, 180, 190, 200, 210, 220, 230, 240, 250, 260, or 270 degrees, or ranges including any two of the foregoing values.

In some embodiments, Arc 1 can have an arc measure of between about 20 degrees and about 270 degrees, between about 80 degrees and about 220 degrees, or about 20, 30, 40, 50, 60, 70, 80, 90, 100, 110, 120, 130, 140, 150, 160, 170, 180, 190, 200, 210, 220, 230, 240, 250, 260, or 270 degrees, or ranges including any two of the foregoing values.

In some embodiments, θ4 can be an angle of between about −10 degrees and about −120 degrees, between about −30 degrees and about −80 degrees, between about −40 degrees and about −90 degrees, between about −60 degrees and about −80 degrees, or about −10, −20, −30, −40, −50, −60, −70, −80, −90, −100, −110, −120 degrees, or ranges including any two of the foregoing values.

Distance X as shown defines the arc length of the distalmost tip portion of the catheter beyond the point where the catheter begins curving in an opposite direction from previous. In some embodiments, Distance X can be between about 2 mm and about 15 mm, between about 2 mm and about 10 mm, or about 2, 3, 4, 5, 6, 7, 8, 9, 10, 11, 12, 13, 14, 15 mm, or ranges including any two of the foregoing values. θ2 is the angle formed between an intersection of the longitudinal axis A1 of a proximal segment and the longitudinal axis A2 of a segment immediately distal to the proximal segment. In some embodiments, θ2 can be an angle of between about 100 degrees and about 180 degrees, between about 140 degrees and about 180 degrees, between about 120 degrees and about 160 degrees, or about 100, 110, 120, 130, 140, 150, 160, 170, 180 degrees, or ranges including any two of the foregoing values. θ3 is the angle formed between an intersection of the longitudinal axis A3 of a proximal segment and the longitudinal axis A4 of a segment immediately distal to the proximal segment, and distal to segments including longitudinal axes A1 and A2. In some embodiments, θ3 can be an angle of between about −10 degrees and about −120 degrees, between about −30 degrees and about −100 degrees, between about −140 degrees and about −180 degrees, between about −50 degrees and about −80 degrees, or about −10, −20, −30, −40, −50, −60, −70, −80, −90, −100, −110, −120 degrees, or ranges including any two of the foregoing values.

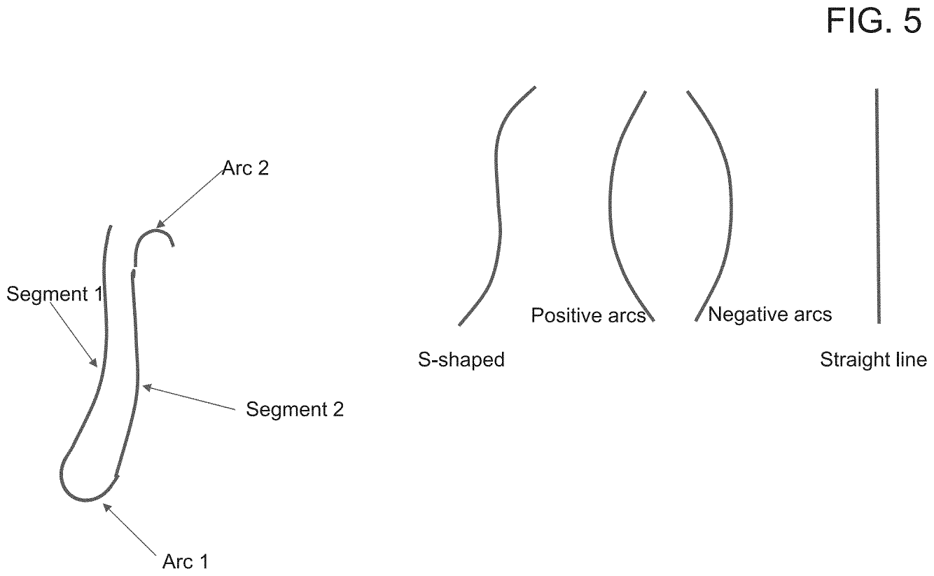

FIG. 5 schematically illustrates additional features of the distal segment/tip portion of the catheter of FIG. 4, according to some embodiments, including a first segment (Segment 1), a first arc (Arc 1), a second segment (Segment 2), and a second arc (Arc 2) arranged proximally to distally. Segment 1 and/or Segment 2 can be a straight (linear) or substantially straight segment, and/or incorporate one, two, or more S-shaped curves, positive arcs, and/or negative arcs, or any combination thereof. For example, in some embodiments Segment 1 can include positive arcs. In some embodiments, Segment 1 can include negative arcs. In some embodiments. Segment 1 can include S-shaped curves. In some embodiments, Segment 2 can include positive arcs. In some embodiments, Segment 2 can include negative arcs. In some embodiments, Segment 2 can include S-shaped curves.

In some embodiments, a distal end of Arc 1 can be axially spaced apart from a proximal end of Arc 2 by a distance of about or less than about 5 mm, 10 mm, 15 mm, 20 mm, 25 mm, 30 mm, 35 mm, 40 mm, 45 mm, 50 mm, or ranges including any two of the foregoing values.

In some embodiments. Segment. 1 can have a length of between about 5 mm and about 65 mm, between about 5 mm and about 40 mm, or about, at least about, or no more than about 5, 10, 15, 20, 25, 30, 35, 40, 45, 50, 55, 60, 65 mm, or more or less, or ranges including any two of the foregoing values.

In some embodiments, Segment 2 can have a length of between about 5 mm and about 65 mm, between about 5 mm and about 40 mm, or about, at least about, or no more than about 5, 10, 15, 20, 25, 30, 35, 40, 45, 50, 55, 60, 65 mm, or more or less, or ranges including any two of the foregoing values.

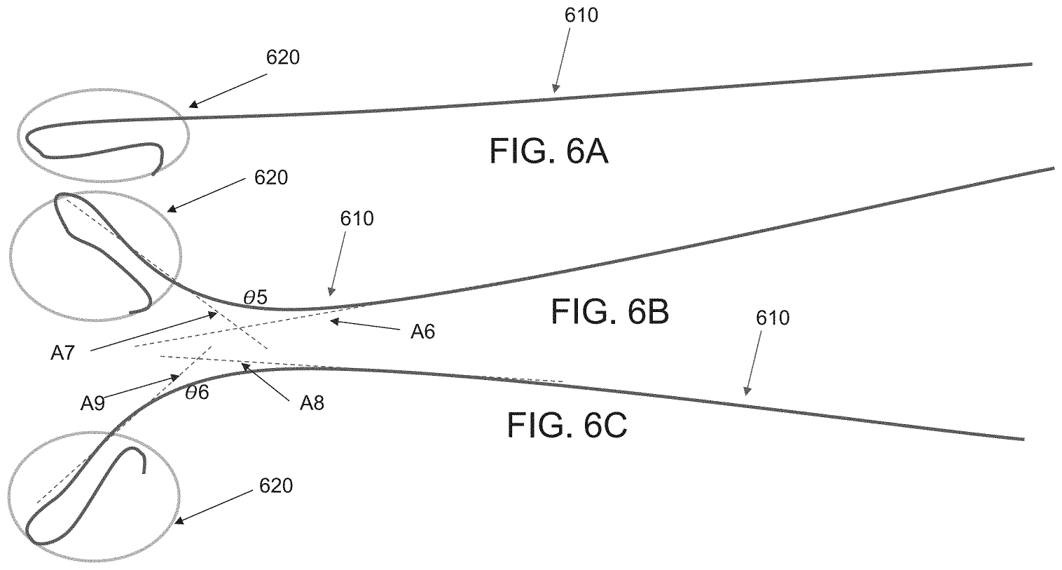

FIGS. 6A-6C schematically illustrate various catheter geometries, according to some embodiments of the invention.

FIG. 6A illustrates a straight or substantially straight proximal segment 610, which directly proceeds a distal segment/tip portion 620 (circled) that can be, for example as previously described.

FIGS. 6B-6C illustrates a proximal segment that includes a straight or substantially straight segment followed by a curved segment, curving in respective different directions. In some embodiments, the curved segment can begin or be entirely within about 20 mm and about 120 mm from the distal end of the catheter (e.g., where contact is made with the aortic cusp), or at about or within about 20 mm, 30 mm, 40 mm, 50 mm, 60 mm, 70 mm, 80 mm, 90 mm, 100 mm, 110 mm, 120 mm from the distal end of the catheter, or ranges including any two of the foregoing values. θ5 in FIG. 6B is the angle formed between an intersection of the longitudinal axis A6 of a proximal segment and the longitudinal axis A7 of a segment immediately distal to the proximal segment. In some embodiments, θ5 can be an angle of between about −100 degrees and about −170 degrees, or about −100, −110, −120, −130, −140, −150, −160, −170 degrees, or ranges including any two of the foregoing values. θ6 in FIG. 6C is the angle formed between an intersection of the longitudinal axis A8 of a proximal segment and the longitudinal axis A9 of a segment immediately distal to the proximal segment. In some embodiments, θ6 can be an angle of between about 100 degrees and about 170 degrees, or about 100, 110, 120, 130, 140, 150, 160, 170 degrees, or ranges including any two of the foregoing values.

In some embodiments, the most proximal curve in the curved versions, such as θ5 in FIG. 6B and θ6 in FIG. 6C for example, can be configured to contact the lesser curve or the greater curve of the ascending aorta. The portion of the catheter that sits across the aortic arch, may be configured to contact the lesser curve of the aorta arch, the greater curve of the aorta arch, or be “free-floating” in the aortic arch depending on manipulation necessary to obtain proper distal catheter position in the aortic cusp of interest,

Conventional catheters generally have one or two purposes: diagnostic angiography and/or use to deliver interventional therapies into coronary/peripheral arteries or structural spaces. Systems and methods including catheters as disclosed herein, in some embodiments, can be advantageously sized and configured to find the nadir or near-nadir of two aortic valve cusps, with the secondary purpose of angiography as clinically necessary.

In some embodiments, an axial length of the total working length of the catheter, or the proximal segment of the catheter (excluding the distal segment/tip portion described above and noted as distal segment 620 in FIGS. 6A-6C) can be, for example, between about 50 mm about 150 mm in length, between about 90 mm and about 140 mm in length, between about 90 mm and about 120 mm in length, or about 50, 60, 70, 80, 90, 100, 110, 120, 130, 140, or 150 mm, or ranges including any two of the foregoing values,

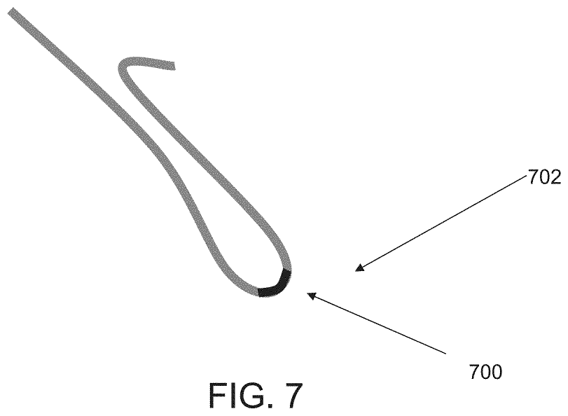

FIG. 7 schematically illustrates the distal end of an elongate member, such as a catheter including one, two, or more radiopaque marker elements. The marker elements 700 could be spaced anywhere along the catheter, including but not limited to a bottom portion 702 of the catheter as illustrated. In one embodiment, a radiopaque marker may include a mixture or alloy of at least two types of metals. Representative examples of biodegradable metals for use in a marker may include, but are not limited to, magnesium, zinc, tungsten, and iron. Representative mixtures or alloys may include magnesium/zinc, magnesium/iron, zinc/iron, and magnesium/zinc/iron, Radiopaque compounds such as iodine salts, bismuth salts, or barium salts may be compounded into certain metallic biodegradable markers to further enhance the radiopacity. Representative examples of biostable metals can include, but are not limited to, platinum and gold.

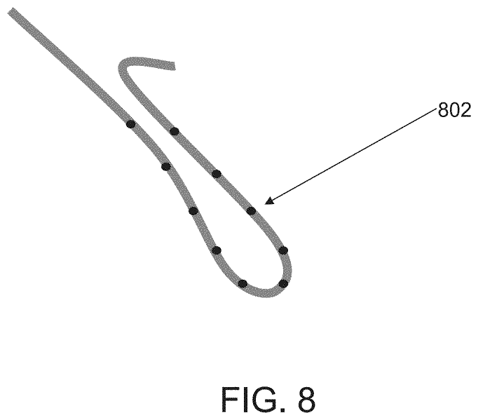

FIG. 8 schematically illustrates the distal end of an elongate member, such as a catheter including a plurality of axially regularly or irregularly spaced-apart side apertures 802 configured to allow for the simultaneous injection of contrast media, such as iodinated contrast. In some embodiments, each side aperture can be spaced apart by a distance of between about 2 mm and about 25 mm, between about 2 mm and about 10 mm, or about 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or ranges including any two of the foregoing values. In some embodiments, the spacing of the side apertures can be variable along certain segments along the length of the capture. For example, the side apertures can be spaced closer together along Arcs 1 and/or 2 and the side apertures can be spaced further apart along Segments 1 and/or 2, In some embodiments, instead or in addition, the side apertures can be oriented in different directions geometrically. For example, the side apertures can be outward oriented in segment 1 and segment 2 and upward oriented (along the top) of Arc 1 as a non-limiting example. In some embodiments, the catheter can also include a distal end-aperture. In some embodiments, the catheter does not include a distal end-aperture.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives failing within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “placing a sample in a transport tube” includes “instructing the placing of a sample in a transport, tube,” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of) and within less than 0.01% of the stated amount.

1-20. (canceled)

21. A catheter system for positioning a transcatheter aortic valve, comprising:
a first catheter configured to be positioned in a first aortic valve cusp;
a second catheter configured to be positioned in a second aortic valve cusp, the second catheter not connected to the first catheter;
a flexible wire configured to be positioned in a third aortic valve cusp;
wherein the first catheter, second catheter, and the flexible wire can be utilized to visualize nadirs of each of the first aortic valve cusp, the second aortic valve cusp, and the third aortic valve cusp in real-time utilizing an imaging device by locating a portion of the first catheter, second catheter, and flexible wire with respect to their proximity to or contact with each of the first aortic valve cusp, the second aortic valve cusp, and the third aortic valve cusp; and
wherein after positioning the first catheter, the second catheter, and the flexible wire each of the first catheter, the second catheter, and the flexible wire are independently movable with respect to each other.

22. The system of claim 21, wherein the first catheter is a pigtail catheter.

23. The system of claim 21, wherein at least one or both of the first catheter and the second catheter comprises a radiopaque marker.

24. The system of claim 21, wherein the second catheter comprises:
a proximal handle;
a proximal segment;
a distal segment and tip,
wherein the distal segment comprises a first portion, a first semicircular arc, a second portion, and a second semicircular arc.

25. The system of claim 24, wherein the first portion comprises a negative arc.

26. The system of claim 24, wherein the first portion comprises a positive arc.

27. The system of claim 24, wherein the second portion comprises a negative arc.

28. The system of claim 24, wherein the second portion comprises a positive arc.

29. The system of claim 24, wherein the first semicircular arc comprise a first central axis of which the first semicircular arc can rotate around, wherein an angle formed by the central axes and a horizontal axis is between about 60 degrees and about 120 degrees.

30. The system of claim 24, wherein the second semicircular arc comprise a second central axis of which the second semicircular arc can rotate around, wherein an angle formed by the central axes and a horizontal axis is between about −30 degrees and about −80 degrees.

31. The system of claim 24, wherein the proximal segment is a substantially straight segment.

32. The system of claim 24, wherein the proximal segment comprises a curved segment having an arc measure of between about 130 degrees and about 170 degrees.

33. (canceled)

34. (canceled)
